(19) United States
(12) Patent Application Publication
(10) Pub. No.: US 2008/0147043 A1
(43) Pub. Date: Jun. 19, 2008

(54) Sheath Assembly Including Coaxial Inner and Outer Sheaths

(76) Inventor: Paul J. Rickards, La Jolla, CA (US)

Correspondence Address: SMITH HOPEN, PA, 180 PINE AVENUE NORTH, OLDSMAR, FL, 34677, US

(21) Appl. No.: 11/611,471

(22) Filed: Dec. 15, 2006

(51) Int. Cl. A61M 25/09; A61M 25/098

(52) U.S. Cl. 604/510; 604/173; 604/164.13

(57) ABSTRACT

A sheath having a tube-in-tube construction includes an outer sheath and in inner sheath. Each sheath has an outer hub at its proximal end, a tubular main body having an endhole at its distal end, and a sidehole formed in the tubular main body. The outer sheath and inner sheath are in coaxial alignment so that they may be rotated relative to one another along a common longitudinal axis. The outer and inner sheaths have a first cooperative position of rotational adjustment where the outer and inner sheath sideholes are in alignment and a second cooperative position of rotational adjustment where the outer and inner sheath sideholes are in misalignment. The sheath enables a user to re-position the sheath from a first to a second blood vessel where said vessels are formed by a fork in a third blood vessel or to reverse direction at a puncture site.

### BACKGROUND OF THE INVENTION

1. Field of the Invention

This invention relates, generally, to angiography sheaths. More particularly, it relates to an angiography sheath having an outer sheath that is coaxial with an inner sheath.

2. Description of the Prior Art

Angiography sheaths are inserted into an artery or a vein at a puncture site. They have a relatively large diameter so that additional instruments may be inserted into and withdrawn from the lumen of the sheath without causing trauma to the artery or vein.

A conventional angiography sheath has an elongate tubular main body and a coaxial hub at its proximal end. The hub has a diameter that is larger than the diameter of the main body of the sheath but the length of the hub is short compared to the length of the main body. The elongate main body, being tubular, terminates in an endhole at its distal end. A radiopaque marker is positioned around the endhole so that the position of the endhole may be seen under fluoroscopy.

At least one injection port is in open communication with the hub and is provided with an open/close valve, typically in the form of a stopcock. The injection port is positioned radially with respect to the hub and thus it is disposed at a right angle to the main body of the sheath.

A guidewire is employed to position the angiography sheath into a desired position. The guidewire is inserted through the puncture site until its free end is positioned at a location where it is desired to position the free end of the angiography sheath. The sheath is inserted through the puncture site and is guided by the guidewire to the desired site. The guidewire is then withdrawn and the sheath is used as a tunnel for additional instruments that are inserted through the puncture site.

Conventional angiography sheaths perform their intended function but they can be difficult to use in those situations where a fork in an artery or vein requires maneuvering of the sheath to avoid positioning an instrument at an undesired location.

Thus there is a need for an improved angiography sheath that facilitates placement of instruments into a desired location.

However, in view of the art considered as a whole at the time the present invention was made, it was not obvious to those of ordinary skill in this art how the identified needs could be met.

### SUMMARY OF INVENTION

The long-standing but heretofore unfulfilled need for an improved sheath is now fulfilled by a new, useful, and nonobvious invention. The novel sheath includes a tube-in-tube construction formed by an outer sheath and an inner sheath. The outer sheath has an outer hub at its proximal end, an elongate tubular main body having an endhole at a distal free end thereof and a sidehole formed in the elongate tubular main body.

The inner sheath has an inner hub at its proximal end, an elongate tubular main body having an endhole at its distal free end, and a sidehole formed in the elongate tubular main body.

The outer sheath and inner sheath are in coaxial alignment with one another so that they may be rotated relative to one another along a common longitudinal axis of rotation. The inner sheath sidehole and the outer sheath sidehole are in alignment with one another in a first cooperative position of rotational adjustment and in misalignment with one another in a second cooperative position of rotational adjustment.

The inner hub is in fluid communication with an injection port.

The outer sidehole is formed in the outer sheath tubular main body at a distance that is about two-thirds or three-fourths of the distance between the outer hub and the endhole of the outer sheath.

A first radiopaque marker is positioned adjacent the outer sidehole and a second radiopaque marker is positioned adjacent the inner sidehole.

A protuberance is formed on an exterior surface of the inner hub and a pair of cooperatively-positioned recesses is formed on an interior surface of the outer hub in diametrically-opposed relation to one another. The protuberance is positioned in a first recess when the respective sideholes formed in the outer and inner sheaths are aligned with one another and the protuberance is positioned in a second recess when the respective sideholes are misaligned with one another.

This structure facilitates the performance of many procedures that are difficult to perform with conventional tools. The novel sheath is advanced into an artery in the closed sidehole position only during initial placement and replacement to avoid shearing injury to the artery at the puncture site. Once in the artery, the sidehole is opened without shearing the wire. Once the desired placement of the wire has been achieved through the sidehole, the sheath is removed completely from the patient in the open sidehole position to avoid shearing the wire off into the artery. The sidehole is then closed outside of the patient and replaced to the desired location over the repositioned guidewire.

### BRIEF DESCRIPTION OF THE DRAWINGS

For a fuller understanding of the invention, reference should be made to the following detailed description, taken in connection with the accompanying drawings, in which:

FIG. 1 is a diagrammatic view of a prior art angiography sheath;

FIG. 2A is a diagrammatic side view of the novel coaxial sheath when the sidehole is open;

FIG. 2B is a diagrammatic end view of the novel coaxial sheath when the sidehole is open;

FIG. 3A is a diagrammatic side view of the novel coaxial sheath when the sidehole is closed;

FIG. 3B is a diagrammatic end view of the novel coaxial sheath when the sidehole is closed;

FIG. 4A is a diagrammatic view depicting the novel coaxial sheath deployed into a common femoral artery and a deep femoral artery that branches from said common femoral artery;

FIG. 4B is a diagrammatic view depicting a guidewire deployed from the novel sheath into a superficial femoral artery when the novel coaxial sheath is deployed in the FIG. 4A configuration;

FIG. 4C is a diagrammatic view depicting the novel sheath when deployed in the common femoral artery and a superficial femoral artery;

FIG. 5A is a diagrammatic view depicting the novel sheath in a common femoral artery in retrograde access;

FIG. 5B is a diagrammatic view depicting how the novel sideholes are used to redeploy the novel sheath from the common femoral artery of FIG. 5A into a superficial femoral artery;

FIG. 6A is a diagrammatic view depicting the novel sheath having its distal end disposed in the abdominal aorta; and

FIG. 6B is a diagrammatic view depicting how the novel sideholes are used to redeploy the distal end of the novel sheath into the contralateral common iliac artery.

### DETAILED DESCRIPTION OF THE PREFERRED EMBODIMENT

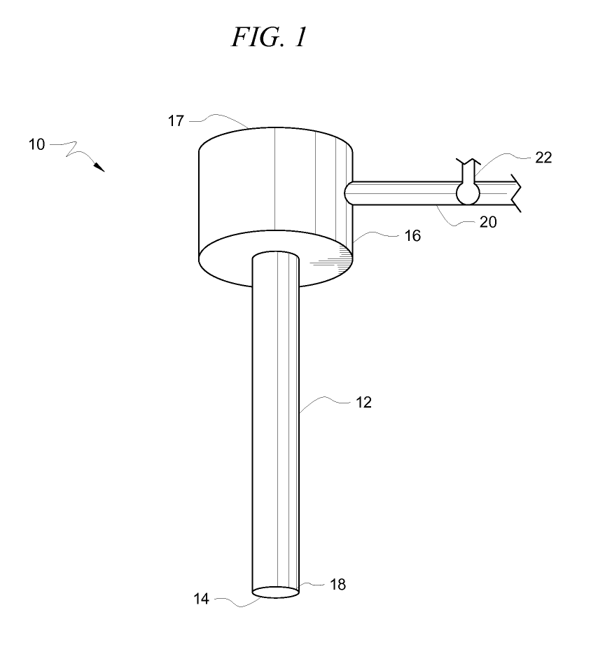

Referring now to FIG. 1, it will there be seen that an angiography sheath of the prior art is denoted as a whole by the reference numeral 10. Sheath 10 includes an elongate tubular main body 12 having an endhole 14 at its distal free end and a hub 16 at its proximal end. A radiopaque marker 18 is provided at said endhole to facilitate its detection under fluoroscopy. An injection port 20 is in fluid communication with hub 16 and extends radially therefrom. Injection port 20 includes an on/off valve in the form of stopcock 22 that allows for aspiration and injection.

A guidewire, not depicted in FIG. 1, is introduced into the lumen of main body 12 at proximal end 17 of hub 16. A sealed one-way entry point is formed in proximal end 17 to prevent distal-to-proximal blood flow but allows placement of various wires and devices into the lumen of main body 12.

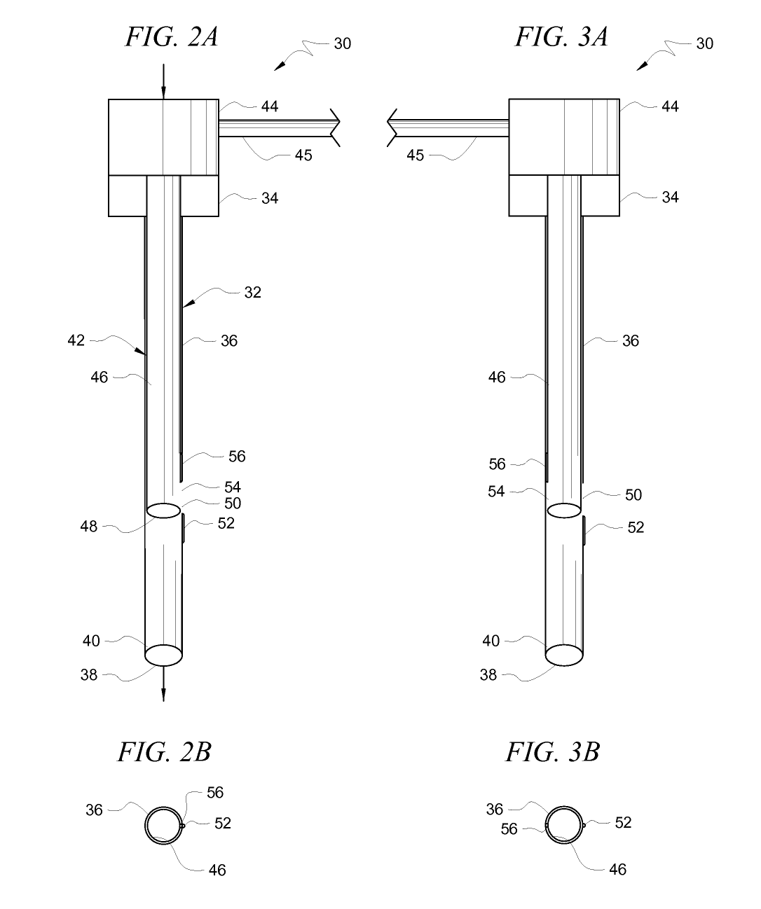

FIGS. 2A and 2B diagrammatically depict novel angiography sheath 30. Novel sheath 30 is a tube-in-tube construction that includes outer sheath 32 having outer hub 34 at its proximal end, elongate tubular main body 36 and endhole 38 at its distal free end. Radiopaque marker 40 is positioned adjacent endhole 38 to facilitate its observation under fluoroscopy.

As with conventional sheaths, a conventional dilator is employed to place the novel sheath into its functional position.

Novel sheath 30 further includes inner sheath 42 having inner hub 44 at its proximal end, said inner hub being in fluid communication with injection port 45, elongate tubular main body 46 and endhole 48 at its distal free end.

As in prior art sheaths, the novel sheath also has a sealed one-way entry point formed in its proximal end to prevent distal-to-proximal blood flow and to allow placement of various wires and devices thereinto.

Outer sheath 32 and inner sheath 42 are in coaxial alignment with one another and inner sheath 42 is snugly positioned within the lumen of outer sheath 32 so that said outer and inner sheaths may be rotated relative to one another along their common longitudinal axis of rotation.

Outer sidehole 50 is formed in outer sheath 32 tubular main body 36 at a distance that is about two-thirds or three-fourths of the distance between outer hub 34 and endhole 38 of said outer sheath. Radiopaque marker 52 is positioned below and adjacent to said outer sidehole 50.

Inner sidehole 54 is formed in inner sheath 42 tubular main body 46 adjacent its endhole 48. Radiopaque marker 56 is positioned above and adjacent to said inner sidehole 54.

As best understood by comparing FIGS. 2A and 2B, outer sidehole 50 is in cooperative alignment with inner sidehole 54 when radiopaque markers 52 and 56 are in rotational alignment with one another. In the side elevational view of FIG. 2A, said alignment is longitudinal. In the end view of FIG. 2B, the alignment is radial. When radiopaque markers 52 and 56 are in such alignment, a sidehole is formed by the alignment of outer sidehole 50 and inner sidehole 54.

FIGS. 3A and 3B depict radiopaque markers 52 and 56 when inner hub 34 has been rotated one hundred eighty degrees from its FIG. 2A and FIG. 2B position, thereby misaligning holes 50 and 54 so that the sidehole created by alignment of said sideholes is closed.

Although not depicted, it should be noted that a small protuberance is formed on an exterior surface of inner hub 44 and two cooperatively-positioned recesses are formed on an interior surface of outer hub 34 in diametrically-opposed relation to one another. The protuberance is therefore positioned in a first recess when the sideholes 50 and 54 are aligned as depicted in FIGS. 2A and 2B and the protuberance is positioned in the second, diametrically-opposed recess when said sideholes are misaligned as depicted in FIGS. 3A and 3B. The entry of the protuberance into a recess is felt by the user so that the user will know by tactile sensation that the sidehole is either fully open or fully closed. The engagement between the protuberance and its recess also prevents unwanted relative rotation between the inner and outer sheaths, thereby preventing kinking or shearing of a guidewire extending therethrough.

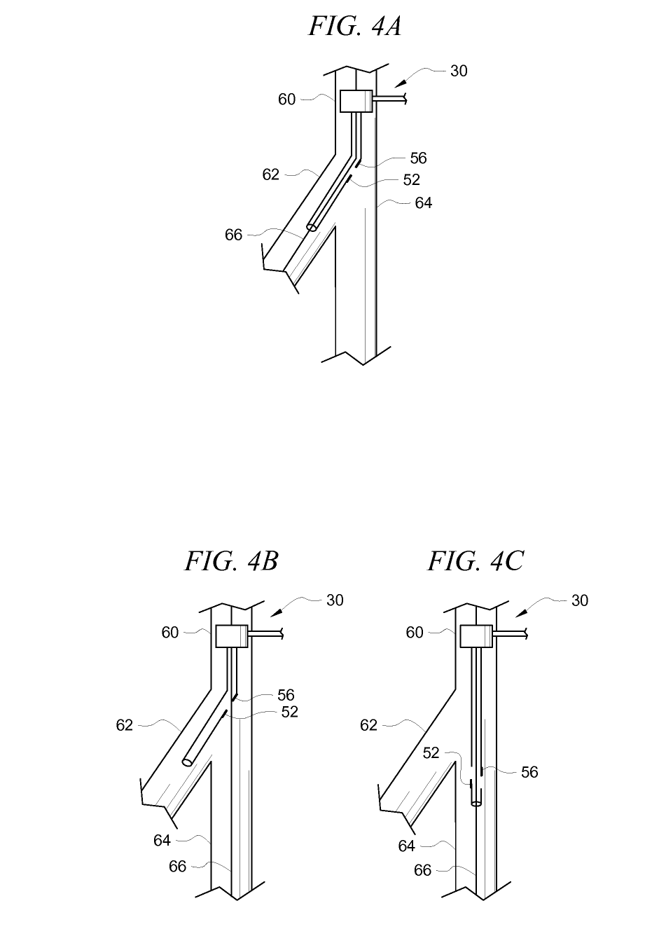

FIGS. 4A, 4B, and 4C provide illustrative examples of how the novel sheath-in-sheath assembly has utility in antegrade access applications. The usual access point in the groin is close to branching vessels in the antegrade and retrograde directions. Accordingly, the operator must pull back the sheath near the vessel entry point to advance the guidewire into the vessel of interest, thereby risking the loss of access by pulling the sheath completely out when doing so. This is particularly problematic when performing antegrade punctures when directing the sheath downstream toward the foot. The wire will frequently enter the deep femoral artery instead of the superficial femoral artery. In an effort to redirect the wire down the superficial femoral artery, the operator must withdraw the catheter or sheath near to the puncture site to redirect the wire down the superficial femoral artery. If the puncture site is close to the fork where the deep femoral and superficial femoral arteries divide from one another, it can be very difficult to direct the wire into the artery of interest and easy to lose access to the artery by pulling the guiding sheath or catheter completely out of the artery. The novel coaxial sheath with its unique sidehole can be used to safely perform these functions while maintaining access to the puncture site as depicted in FIGS. 4A-C. Moreover, due to the locking coaxial hub, the sheath can be transformed back into a traditional endhole sheath to continue the procedure without opening a new catheter or sheath.

FIGS. 4A-C depict a common femoral artery 60 that branches into a deep femoral artery 62 and a superficial femoral artery 64.

In FIG. 4A, guidewire 66 and sheath 30 are both disposed in deep femoral artery 62. The sidehole is open. Instead of needing to retract the sheath and guidewire as required by prior art sheaths, only guidewire 66 needs to be retracted, with the novel sheath remaining in its FIG. 4A position. After guidewire 66 is retracted to a point upstream of the sidehole, said guidewire is then fed through the sidehole into superficial femoral artery 64 as depicted in FIG. 4B. Sheath 30 is then removed from the artery while maintaining wire access to the superficial femoral artery. The sidehole is closed outside of the patient by rotating the inner hub 44 one hundred eighty degrees and sideholes 50, 54 are misaligned. The sheath is then replaced into superficial femoral artery 64 by following the guidewire.

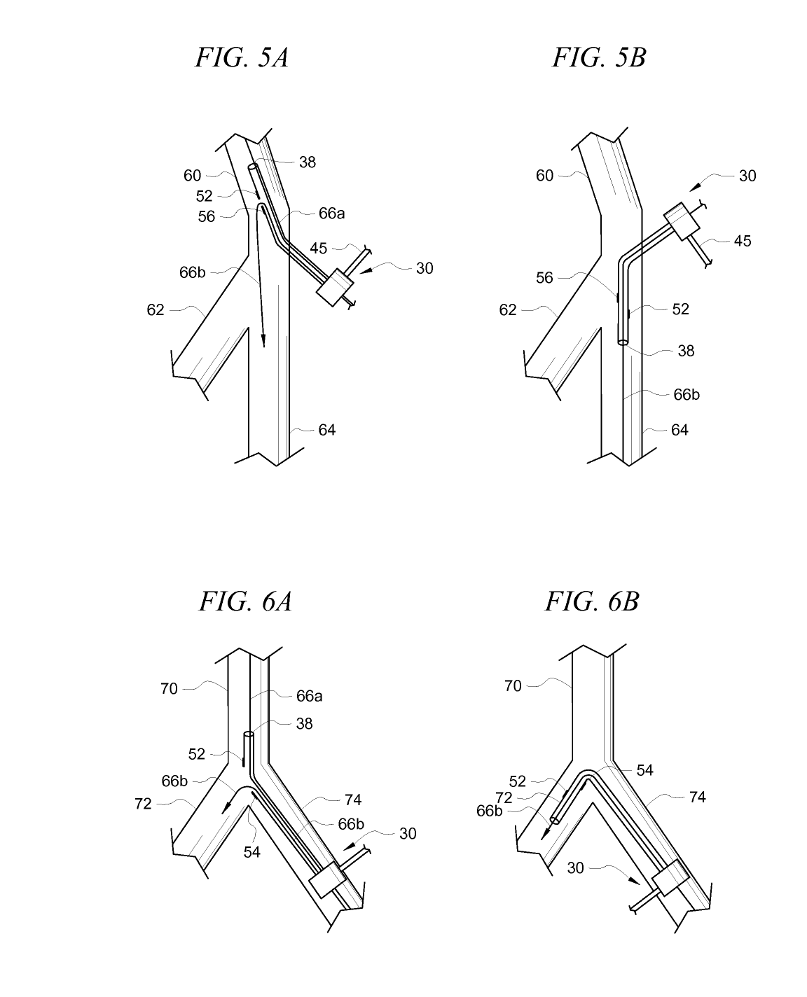

Referring now to FIGS. 5A and 5B, it is there depicted how the novel device facilitates reverse direction of access. As in FIGS. 4A-C, FIGS. 5A-B depict a common femoral artery 60 that branches into a deep femoral artery 62 and a superficial femoral artery 64.

With conventional single endhole access sheaths, the operator is limited to a single direction during the procedure. If the puncture is made going from groin to abdomen, it is called a retrograde puncture and access. If the puncture is made angling down from the groin to foot it is called an antegrade puncture. Once the decision is made to go a particular way, it is very difficult and frequently impossible to change directions and go the other way without making an entirely separate puncture which leaves the first puncture site unplugged. This is problematic if the patient is taking blood thinners for anticoagulation because severe bleeding from the initial access point may result after removing the sheath or catheter. With the novel coaxial sidehole sheath, it is possible to reverse the direction of access from the original puncture site, simply by placing a second guidewire through the sidehole in the opposite direction to the initial access. Once the second guidewire has been advanced down the vessel in the opposite direction, the sheath is removed from the artery, the sidehole closed, and sheath 30 is replaced over the second guidewire down the other direction (into superficial femoral artery 64) as depicted in said FIGS. 5A and 5B.

More particularly, as depicted in FIG. 5A, where sheath 30 is in retrograde access, a second guidewire 66b is advanced through the sidehole adjacent to first guidewire 66a in the opposite direction relative to the direction of sheath 30. The sheath is then removed from the artery over both guidewires 66a and 66b, the sidehole is closed outside of the body of the patient, and the sheath is replaced in the opposite direction over second guidewire 66b as depicted in FIG. 5B. First guidewire 66a which is now outside of the sheath is removed altogether leaving the sheath in the antegrade direction over second guidewire 66b.

FIGS. 6A and 6B depict novel sheath 30 in use to cross the abdominal aortic bifurcation. The abdominal aorta is denoted 70, the right common iliac artery is denoted 72, and the left common iliac artery is denoted 74.

FIG. 6A depicts retrograde access up through the iliac bifurcation over first guidewire 66a. Radiopaque markers 52 and 54 are aligned, indicating that the sidehole is open. Endhole 38 of novel sheath 30 is in the abdominal aorta 70 and a second guidewire 66b is being advanced into the right common iliac artery 72 through the sidehole.

With a traditional single endhole sheath, the catheter simply points straight ahead as depicted in said FIG. 6A. Due to the sharp fork of the abdominal aortic bifurcation, it is nearly impossible to cross over to the other side of the body without the use of a new catheter. With the sidehole design and with variable lengths of the sheath, the operator simply advances the sidehole to the bifurcation point and guides a second wire out of the sideport down the other side. The sheath is then removed from the patient over both guidewires. The sideport is closed by rotating hub 44 so that radiopaque markers 52, 54 are misaligned, thereby indicating closing of the sidehole as depicted in FIG. 6B, and the same sheath is re-advanced to the contralateral side over second guidewire 66b as depicted in FIG. 6B. First guidewire 66a is now outside sheath 30 and is removed leaving the sheath down the opposite side over the second guidewire. FIG. 6B depicts the final result after removal of first guidewire 66a.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A sheath, comprising:
a tube-in-tube construction including an outer sheath and in inner sheath;
said outer sheath having an outer hub at its proximal end;
said outer sheath having an elongate tubular main body having an endhole at a distal free end of said elongate tubular main body;
a sidehole formed in said elongate tubular main body of said outer sheath;
said inner sheath having an inner hub at its proximal end;
said inner hub having an elongate tubular main body and an endhole at its distal free end;
a sidehole formed in said elongate tubular main body of said inner sheath;
said outer sheath and inner sheath being in coaxial alignment with one another so that said outer and inner sheaths may be rotated relative to one another along a common longitudinal axis of rotation;
said outer sheath and said inner sheath having a first cooperative position of rotational adjustment where said inner sheath sidehole and said outer sheath sidehole are in alignment with one another; and
said outer sheath and said inner sheath having a second cooperative position of rotational adjustment where said inner sheath sidehole and said outer sheath sidehole are in misalignment with one another.

2. The sheath of claim 1, further comprising:
said inner hub being in fluid communication with an injection port.

3. The sheath of claim 1, further comprising:
said outer sidehole formed in said outer sheath tubular main body at a distance that is about two-thirds or three-fourths of the distance between said outer hub and said endhole of said outer sheath.

4. The sheath of claim 3, further comprising:
a radiopaque marker positioned adjacent said outer sidehole.

5. The sheath of claim 4, further comprising:
a radiopaque marker positioned adjacent said inner sidehole.

6. The sheath of claim 1, further comprising:
a protuberance formed on an exterior surface of said inner hub;
a pair of cooperatively-positioned recesses formed on an interior surface of said outer hub in diametrically-opposed relation to one another;
said protuberance being positioned in a first recess when the respective sideholes formed in said inner and outer sheaths are aligned with one another;
said protuberance being positioned in a second recess when the respective sideholes formed in said inner and outer sheaths are misaligned with one another.

7. A method for inserting a sheath into an artery, comprising the steps of:
providing a sheath having a tube-in-tube structure where each tube has a sideport formed therein;
forming an open sideport in said sheath by relatively rotating the tubes that collectively form the sheath until their respective sideports are in alignment with one another;
closing said open sideport in said sheath by relatively rotating the tubes that collectively form the sheath until their respective sideports are in misalignment with one another;
advancing the sheath into an artery in the closed sidehole position only during initial placement and replacement to avoid shearing injury to the artery at the puncture site;
opening the sidehole without shearing the wire after the sheath is in the artery;
removing the sheath completely from the patient in the open sidehole position to avoid shearing the wire off into the artery;
closing the sidehole outside of the body of the patient and replacing the sheath to the desired location over the repositioned guidewire after a desired placement of the guidewire has been achieved through the sidehole.
